(19) United States
(12) Patent Application Publication
(10) Pub. No.: US 2015/0164434 A1
(43) Pub. Date: Jun. 18, 2015

(54) METHOD AND APPARATUS FOR DETECTING A CRITICAL SITUATION OF A SUBJECT

(71) Applicants: VIGILIO, Evry (FR); UNIVERSITE JOSEPH FOURIER, Grenoble (FR)

(72) Inventors: Norbert Noury, Grenoble (FR); Jean-Eric Lundy, Thiais (FR)

(21) Appl. No.: 14/631,110

(22) Filed: Feb. 25, 2015

Related U.S. Application Data
(63) Continuation of application No. 12/746,399, filed on Jun. 4, 2010, now abandoned.

(30) Foreign Application Priority Data
Dec. 6, 2007 (FR) 0759618

(51) Int. Cl. A61B 5/00; A61B 5/0205; A61B 5/11; A61B 5/07

(52) U.S. Cl. CPC A61B 5/7282; A61B 5/076; A61B 5/02055; A61B 5/7264; A61B 5/1118; A61B 5/7246; A61B 5/746; A61B 5/7405; A61B 5/1117; A61B 5/02405

(57) ABSTRACT

Embodiments of the invention relate to a method for detecting the critical situation of a subject that comprises measuring a physiological parameter of the subject. The method includes (a) measuring the physiological parameter; (b) calculating a variation rate of the physiological parameter; (c) determining a synthesis variable for the physiological parameter by taking into account both the value of the parameter and the variation rate thereof; and (d) detecting a critical situation from the synthesis variable. Steps (a) to (c) may be implemented for at least two physiological parameters, and the method may further include determining a global decision variable from the different synthesis variables, the critical situation being detected during step (d) from the global decision variable. Embodiments of the invention can be used for improving the detection of accidents or health problems, particularly for the elderly at their places of residence.

### CROSS-REFERENCE TO RELATED APPLICATION

The present application is a continuation of U.S. application Ser. No. 12/746,399, filed on Jun. 4, 2010, titled “Method and Apparatus for Detecting a Critical Situation of a Subject,” which is a National Stage Entry of PCT/EP08/066736, filed on Dec. 3, 2008, which claims priority from French application FR 0759618, filed on Dec. 6, 2007, the contents of each of which are incorporated herein by reference.

### FIELD OF THE INVENTION

The present invention relates to a process and device for detecting a critical situation of a subject.

Detection of a critical situation of a subject can prove useful in numerous cases, for example in the case of aged or infirm persons. Aged persons often live alone and in the event of an accident, such as a fall, they can be incapable of asking for help. These people may also encounter a health problem during their daily life or when asleep. It is therefore useful to provide automatic detection systems of critical situations, which can be coupled to alarm and/or call systems.

There are shock sensors worn by people, which give off an alarm if these people are subjected to shock. However, for this type of detection system to be efficacious, the person would have to wear a large number of these sensors, in principle one on each salient body part, so this is not a practical solution. There are also verticality sensors which detect whether a person is lying down or upright. However, with this type of sensor, it is impossible to know whether a person is lying down voluntarily (rest, nap) or if he has been victim of a fall. The performance of a fall sensor can thus be appreciated only by factors of sensitivity (capacity of the system to detect all falls) and specificity (capacity of the system to detect falls only). Yet, a fall can take multiple forms (front, rear, side, straight or with rotation of the body, ending up in a prostrate or seated position, etc.).

It is therefore almost impossible for a fall sensor to discriminate exhaustively between real falls and false falls. Also, the same fall motion, even the simplest (for example a rearwards fall after slipping) can turn out to be variable according to context and subject. Consequently, it is not possible to attain total sensitivity and total specificity with fall detection means which are reasonable in terms of their bulk, complexity and cost. Also, this type of equipment is not capable of detecting a critical situation especially due to a health problem when the person is lying down, for example.

The same type of problem can occur with a cardiac rhythm sensor, where a situation can be normal in a certain context and alarming in another.

It is also known to further improve the performance of a sensor such as a fall sensor by combining the information it puts out with geographical or time information (GPS geolocalisation, duration of immobilisation, . . . ). Document EP 0 877 346 is a particular example. But the system described in this document involves a certain technical complexity and associated costs.

The aim of the present invention is to eliminate these limitations of the prior art and propose a process and equipment which will, without increasing technical complexity or cost, bring about improved discrimination due to decision strategy based on measuring physiological parameters to detect a critical situation of a subject.

### BACKGROUND OF THE INVENTION

For this purpose, a process for detecting a critical situation of a subject by measuring a physiological parameter on the subject is provided according to embodiments of the invention comprising the following steps:

(a) measuring the physiological parameter by means of an embedded sensor on the subject,

(b) calculating a variation rate of the physiological parameter,

(c) determining a synthesis variable for the physiological parameter by taking into account at the same time the value of the parameter and its variation rate, and

(d) detecting a critical situation from this synthesis variable.

Such a process reliably detects a critical situation, but without so much increasing the cost and complexity of the device, the decision part being made by a data-processing unit already present in such devices and requiring no supplementary equipment.

Advantageously, though optionally, the invention comprises at least one of the following characteristics:

the process comprises implementing steps (a) to (c) for at least two physiological parameters, and further comprises the following step:

(c′) determining a global decision variable from the different synthesis variables, the critical situation being detected in step (d) from this global decision variable.

the physiological parameters are selected from the following parameters: cardiac frequency, respiratory frequency, and temperature of the subject, level of activity of the subject, glycaemia of the subject.

determination of the global decision variable is done by a weighted combination of synthesis variables determined for each of the physiological parameters.

detection of a critical situation comprises comparison of the global decision variable with one or more threshold values.

determination of the synthesis variable for a physiological parameter comprises comparison of the value of the parameter with one or more threshold values.

determination of the synthesis variable for a physiological parameter comprises comparison of the variation rate of said physiological parameter with one or more threshold values.

the value of the parameter is compared to two threshold values to determine if the value of the parameter is low, normal or high, and the variation rate of the parameter is compared to a threshold value to determine if the variation rate of the parameter is weak or strong.

the value of the synthesis variable is:

(1) higher if the value of the parameter is high than if the value of the parameter is normal,

(2) higher if the value of the parameter is low than if the value of the parameter is normal,

(3) higher if the variation rate of the parameter is strong than if the variation rate of the parameter is weak.

the process further comprises the following step:

(e) remote transmission via a wireless link of the existence of said critical situation.

Embodiments of the invention also propose detection equipment of a critical situation of a subject comprising:

at least one embedded sensor capable of measuring a physiological parameter, and

a processing unit capable of receiving the values measured by the sensor or sensors and for calculating a variation rate of the physiological parameter or of each of the physiological parameters, and determining a synthesis variable for the physiological parameter or each of the physiological parameters by taking into account at the same time its value and its variation rate, for the parameter or for at least one of the parameters.

Preferred, though non-limiting, aspects of this equipment are the following:

the processing unit is capable of executing the process such as defined hereinabove in steps (b), (c), (c′) and (d).

the equipment is fully embedded on the subject and further comprises remote transmission means of information relative to the existence of a critical situation.

the equipment comprises an embedded device including the sensors and an external station housing at least part of the processing unit and further comprising remote transmission means of information relative to the existence of a critical situation.

### BRIEF DESCRIPTION OF THE SEVERAL VIEWS OF THE DRAWINGS

Other characteristics, aims and advantages of the present invention will emerge from the following detailed description, in relation to the attached diagrams given by way of non-limiting examples in which:

FIG. 1 is a schematic representation of equipment according to the invention;

FIG. 2 is a schematic diagram of a process according to the invention; and

FIG. 3 is another schematic diagram of a process according to the invention.

### DETAILED DESCRIPTION

The following description describes a remote surveillance system of persons based on one or more embedded detectors sensitive to movements of the body of the person (especially accelerometers) and to the vital variables of the person (for example a sensor for cardiac rhythm, respiratory rhythm, arterial pressure, etc. and more generally of any variable sensed on the human body and likely to have a correlation with the state of health). Throughout the document “physiological parameters” stands for all variables measured by the detection equipment.

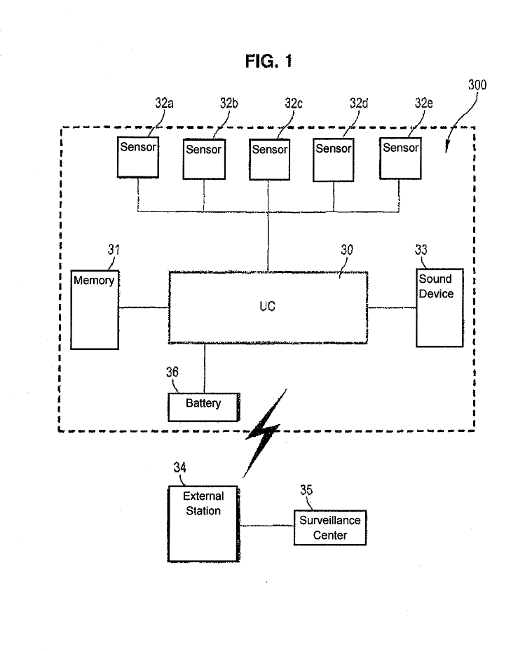

In reference to FIG. 1, fall detection equipment 300 according to the invention comprises a central processing unit 30 linked to a memory 31, the whole being made for example in the form of a microcontroller and powered by an onboard power source such as a battery 36. The memory 31 especially contains the detection program, as will be described below, and the associated data. A set of embedded sensors 32a to 32e is connected to inputs of the central unit 30, each being capable of measuring a physiological parameter of the subject.

For example, inclination sensors and movement sensors can be utilised to measure the inclination and constituent of the acceleration of gravity along the torso and leg of the person. So for example, if the person is leaning against a wall and his torso stays vertical, the sensor placed on the legs could indicate if the person falls.

More generally, such sensors 32a to 32e can be provided for measuring, for example:

cardiac frequency,

respiratory frequency,

temperature of the subject, or the relation between the temperature of the subject and ambient temperature,

the level of activity of the subject (determined for example by the same sensors as those used to detect a fall),

glycaemia, for example by means of a glucose sensor, such as a sensor commercially available from one of the companies Medtronic, Abbott and Dexcom.

A sound device 33 is connected to the central unit 30 so that the equipment can emit a sound alert signal when a fall is detected. In the same way, the detection equipment 300 is connected via a transmitter device, not shown here, to an external station 34 which is responsible for transmitting the alert signal to a remote surveillance centre 35 via hertzian or telephone connection or via internet. When it detects no fall, the central unit 30 can deliver a normal situation signal at regular time intervals to indicate that the equipment is operating. The central unit 30 can also supply at regular time intervals or not a signal indicating that the equipment is being worn by the person and that the latter is being monitored.

In one embodiment, processing of measurements originating from the sensors is carried out at the external station 34, the control unit 30 in this case simply serving to transmit measurements of the sensors 32a to 32e to the external station 34. It can be provided in this case that the external station 34 has a sound device designed to emit a sound alert signal more powerful than that of the detection equipment 300 in the event of a detected fall.

Accordingly, either the equipment is fully embedded on the person and comprises remote transmission means of information relative to the existence of a critical situation, or the equipment comprises an embedded device including sensors and an external station housing at least part of the calculator and further comprising remote transmission means of information relative to the existence of a critical situation.

According to the invention, a critical situation of a subject wearing the equipment is detected on the basis of a combination of measurements of the device. Using this combination, the equipment can detect whether an alert should be triggered or not.

More particularly, it is provided that measurement of a physiological parameter supplied by the detection equipment does not necessarily have the same weight according to the physiological parameter, the value of this parameter and the variation rate of this parameter.

The process according to embodiments of the invention, carried out by the central unit 30 or by the external station 34 (or even shared between them), comprises the following steps:

measuring each of the physiological parameters,

calculating a variation rate of each of the physiological parameters,

determining a synthesis variable for each of the physiological parameters by taking into account at the same time the value of the parameter and its variation rate,

in the event where several physiological parameters are considered, determining a global decision variable for all of the latter, and

detecting a critical situation from this global decision variable.

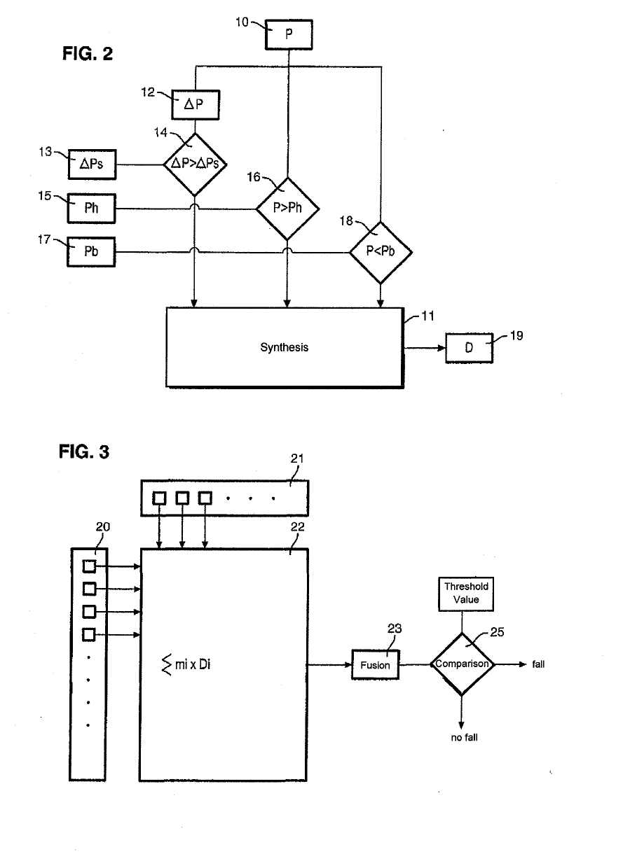

In reference to FIG. 2, a decision process according to the invention will now be detailed. Each physiological parameter p is defined by its instantaneous value p and its tendency Δp (absolute value of the variation of the value p between two measuring times separated by a fixed interval). A measuring validation process is advantageously provided, for example by validating a measurement only if its value correlates to the preceding values of this measurement.

Two threshold values ph (high) and pb (low) are defined for each parameter p, as well as a threshold value Δps for the tendency Δp.

In this way, a comparison is made at 14 between Δp and the threshold Δps, a comparison is made at 16 between the value p and the threshold ph, and a comparison is made at 18 between the value p and the threshold pb.

For example, if the physiological parameter is the body temperature (normal value 37° C.), the three threshold values of the parameter can be:

ph=40° C.

pb=35° C.

Δps=1° C./hour.

These thresholds can be determined by expertise of one or more physicians utilising their knowledge of human physiology and their practical experience. These thresholds can be also determined by statistical analysis on data recorded on a subject (intra-subject analysis) or on a group of subjects belonging to a target population (inter-subject analysis). This statistical analysis can be carried out once only, or else periodically, to take into account the evolution of the subject or of the target population (seasonal variations, tendencies due to aging).

Advantageously, some of these thresholds, in the configuration of the system, can be adjusted as a function of certain known parameters, physiological or not, of the subject, such as age, sex, mass index, antecedents, lifestyle, various environmental factors, etc. For example, for a person of high mass index, the glycaemia threshold values will be higher, the cardiac and respiratory frequency tendency thresholds will be higher, etc.

These threshold variables can be periodically updated manually or automatically in the event where these known parameters vary, and in the event where the system is capable of recognising these variations (age for example).

From the results of these comparisons, the process calculates a synthesis value of the physiological variable D. The aim of this value is to supply simplified information of the state of the physiological parameter. Throughout the document, it is agreed that the higher the synthesis value D, the more disturbing the state of the physiological variable.

The values of a parameter p can be compared relative to its thresholds ph and pb and its tendency Δp relative to the threshold Δps:

If p<pb, then p is too low;

If p>ph then p is too high;

If pb<p<ph then p is normal;

If Δp>Δps then the tendency Δp is strong, otherwise it is weak.

A synthesis matrix for the parameter p is then worked out in step 11, as follows for example:

| | Low | Normal | High |
|---|---|---|---|
| Weak | 0.5 | 0 | 0.5 |
| Strong | 1 | 0.5 | 1 |

in which the lowest synthesis value (D=O) is worked out if the level of the parameter and its variation do not signify trouble, and the highest synthesis value (D=1) if the parameter moves too far away from normal at the same time by its value and its tendency, and can therefore signify trouble.

Accordingly, from a synthesis matrix and comparisons made in steps 14, 16 and 18, step 11 outputs the synthesis value D.

For example, if the physiological parameter is cardiac frequency (nominal value of 70 beats per minute or BPM at rest for any given person) with:

ph=100 BPM

pb=40 BPM

Δps=15 BPM/minute

if at any given instant p=60 BPM with Δp=20 BPM/min is registered, the parameter p of a level of synthesis D=0.5 will be assigned.

At any given instant, the values of n physiological parameters are known:

<pi>=[p1, p2, . . . pi . . . pn]

and their tendencies:

<Δpi>=[Δp1, Δp2, . . . Δpi . . . Δpn]

For each of these parameters, thresholds phi and pbi are fixed, as well as tendency thresholds Δpsi.

The synthesis values <Di>=[D1, D2, . . . Di . . . Dn] are therefore calculated.

As has been pointed out, the parameters taken into consideration can especially be cardiac frequency, respiratory frequency, temperature of the subject, or the relation between the temperature of the subject and ambient temperature, the level of activity of the subject (determined for example by accelerometers) and glycaemia.

In reference to FIG. 3, a weight mi is assigned to each synthesis value Di of a parameter pi. So, in block 21 a weight vector mi <mi>=[m1, m2, . . . mi . . . mn] is assigned, for example, such as Σmi=1 (the sum of all weights is equal to 1) and in block 20 a vector of the synthesis value di, <Di>=[D1, D2, . . . Di . . . Dn] is assigned. These two blocks are linked to block 22, in which a fusion value denoted Fusion is calculated, such as for example:

Fusion=Σmi*Di

This value constitutes a global decision variable representative of the state of the synthesis values of the different physiological parameters, by taking into account their importance due to weighting; this information helps decide whether a critical situation is detected or not.

Therefore, at block 25 the fusion value is compared to one or more threshold values with the aim of detecting a critical situation and triggering or not an alarm signal.

For example, it can be established by agreement that if Fusion>0.5 then this means the presence of a critical situation.

Alternatively, two thresholds on fusion can be envisaged, for example:

If 0.3<Fusion<0.7, then this means the presence of a situation of major concern (orange alert);

and if 0.7<Fusion then this means the presence of a critical situation (red alert).

Here is a concrete example with cardiac frequency (Fc in BPM), respiratory frequency (Fr in nb/mn) and body temperature (T in ° C.) as parameters:

| | mini | maxi | low | high | tendency | |
|---|---|---|---|---|---|---|
| parameter | value | value | threshold | threshold | threshold | weight |
| Cardiac | 50 | 180 | 60 | 90 | 13 | 0.5 |
| freq. (Fc) | | | | | | |
| respiration | 1 | 10 | 4 | 7 | 0.9 | 0.3 |
| (Fr) | | | | | | |
| Temperature | 35 | 42 | 36 | 39 | 0.7 | 0.2 |
| (T) | | | | | | |

| | | case 1 | case 2 | case 3 |
|---|---|---|---|---|
| parameter | Fc | 70 | 50 | 100 |
| parameter | | 0 | 1 | 1 |
| threshold | | | | |
| tendency | DFc | 5 | 5 | 15 |
| tendency | | 0 | 0 | 1 |
| threshold | | | | |
| Fc decision | D1 | 0 | 0.5 | 1 |
| parameter | Fr | 5 | 4 | 3 |
| parameter | | 0 | 0 | 1 |
| threshold | | | | |
| tendency | DFr | 0.5 | 1 | 1 |
| tendency | | 0 | 1 | 1 |
| threshold | | | | |
| Fr decisions | D2 | 0 | 0.5 | 1 |
| parameter | T | 36.4 | 35 | 41 |
| parameter | | 0 | 1 | 1 |
| threshold | | | | |
| tendency | DT | 0.5 | 2 | 2 |
| tendency | | 0 | 1 | 1 |
| threshold | | | | |
| decision T | D3 | 0 | 1 | 1 |
| Fusion | | 0.00 | 0.60 | 1.00 |
| Alert | | no | orange | red |

in the first case, there is nothing abnormal.

in the second case, a first alert level (orange) is reported, since cardiac frequency is low, respiratory frequency is normal though has a tendency to drop, and body temperature is low.

the last case tends towards tachycardia (already high cardiac frequency continuing its acceleration); on the contrary low respiration does not follow this rhythm and there is also risk of an infectious outbreak by the high temperature.

Many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A process for detecting a critical situation of a subject, the process comprising the following steps:
(a) measuring at least one physiological parameter by means of an embedded sensor on the subject;
(b) calculating a variation rate of each physiological parameter;
(c) determining a synthesis value for each physiological parameter by applying a synthesis matrix to the value of the parameter and its variation rate, wherein the synthesis matrix is a predetermined matrix of values correlating low, normal, and high values of physiological parameters with weak and strong variation rates, and wherein the synthesis value represents a single value that takes into account both the physiological parameter and its variation rate;
(d) calculating a global decision value by adding together the synthesis value determined for each physiological parameter; and
(e) determining whether a critical situation exists based on the global decision value.

2. The process as claimed in claim 1, wherein the physiological parameters are selected from the group consisting of cardiac frequency, respiratory frequency, temperature of the subject, level of activity of the subject, and glycaemia of the subject.

3. The process as claimed in claim 1, wherein determination of the global decision variable is done by weighted combination of the synthesis variables determined for each of the physiological parameters.

4. The process as claimed in claim 1, wherein determining whether a critical situation exists comprises comparing the global decision variable with one or more threshold values.

5. The process as claimed in claim 1, wherein determination of the synthesis variable for a physiological parameter comprises comparison of the value of the parameter with one or more threshold values.

6. The process as claimed in claim 1, wherein determination of the synthesis variable for a physiological parameter comprises comparison of the variation rate of said physiological parameter with one or more threshold values.

7. The process as claimed in claim 5, wherein determination of the synthesis variable for a physiological parameter comprises comparison of the variation rate of said physiological parameter with one or more threshold values, wherein the value of the parameter is compared to two threshold values to determine if the value of the parameter is low, normal or high, and wherein the variation rate of the parameter is compared to a threshold value to determine if the variation rate of the parameter is weak or strong.

8. The process as claimed in claim 7, wherein the value of the synthesis variable is:
higher in an instance in which the value of the parameter is high as compared to an instance in which the value of the parameter is normal,
higher in an instance in which the value of the parameter is low as compared to an instance in which the value of the parameter is normal,
and
higher in an instance in which the variation rate of the parameter is strong as compared to an instance in which the variation rate of the parameter is weak.

9. The process as claimed in claim 1, further comprising the following step:
(f) remotely transmitting via wireless link of the existence of said critical situation.

10. An apparatus for detecting a critical situation of a subject comprising:
at least one embedded sensor capable of measuring a physiological parameter, and
a processing unit configured to receive the values measured by the sensor or the sensors, to calculate a variation rate of the physiological parameter or of each of the physiological parameters, and to determine a synthesis variable for the physiological parameter or each of the physiological parameters by applying a synthesis matrix to the value of the parameter and its variation rate, wherein the synthesis matrix is a predetermined matrix of values correlating low, normal, and high values of physiological parameters with weak and strong variation rates, and wherein the synthesis value represents a single value that takes into account both the physiological parameter and its variation rate,
wherein the processing unit is further configured to calculate a global decision value by adding together the synthesis value determined for each physiological parameter and to determine whether a critical situation exists based on the global decision value.

11. The apparatus as claimed in claim 10, wherein the apparatus is fully embedded on the subject and further comprises a transmitter configured to remotely transmit information relative to the existence of a critical situation.

12. The apparatus as claimed in claim 10, further comprising an embedded device including the sensors and an external station housing at least part of the processing unit, and further comprising a transmitter configured to remotely transmit information relative to the existence of a critical situation.
